(19) United States
(12) Patent Application Publication
(10) Pub. No.: US 2012/0253051 A1
(43) Pub. Date: Oct. 4, 2012

(54) PROCESS FOR THE PREPARATION OF ROPINIROLE AND SALTS THEREOF

(75) Inventors: Theoharis V. Koftis, Thessaloniki (GR); Ioanna Georgopoulou, Thessaloniki (GR); Ravikant R. Soni, Thessaloniki (GR); Panagiota Mandalou, Thessaloniki (GR); Elli Alexandraki, Thessaloniki (GR)

(73) Assignee: PHARMATHEN S.A., PALLINI-ATTIKIS (GR)

(21) Appl. No.: 13/515,881

(22) PCT Filed: Dec. 16, 2009
(86) PCT No.: PCT/EP09/09023

§ 371 Date: Jun. 14, 2012

(51) Int. Cl. C07D 209/34

(52) U.S. Cl. 548/486

(57) ABSTRACT

The present invention relates to an improved process for the preparation of Ropinirole and pharmaceutical acceptable salts or derivatives thereof, in particular to a process for large scale production of Ropinirole and salts thereof in high yield and high purity and pharmaceutical preparations containing said compounds.

### TECHNICAL FIELD OF THE INVENTION

The present invention relates to an improved process for the preparation of Ropinirole and pharmaceutical acceptable salts or derivatives thereof and in particular to a process for large scale production of Ropinirole or salts thereof and pharmaceutical preparations containing said compounds.

### BACKGROUND OF THE INVENTION

Ropinirole is a known non-ergoline dopamine agonist, a selective D2-agonist, employed for the treatment of Parkinson's disease with much less undesirable side effects than other dopaminergic agents.

Ropinirole is chemically designated as 4-[2-(di-n-propylamino) ethyl]-1, 3-dihydro-1H-indole-2-one and is used in the form of hydrochloride salt. Ropinirole hydrochloride is presented by the following structure of Formula I.

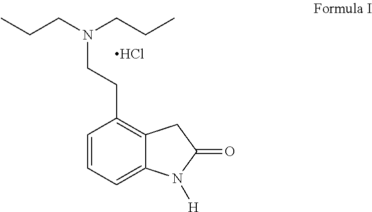

Various methods are already known for the preparation of Ropinirole or salt thereof due to its useful properties. Prior art processes for the preparation of Ropinirole hydrochloride present the disadvantage of non-satisfactory yield of the product. Furthermore, the compound often comprises significant amounts of impurities.

EP-B-0113964 discloses a process wherein 2-methyl-3-nitrophenylethyl-N, N-di-n-propylamine is subjected to addition, hydrolysis, decarboxylation and reductive cyclization to obtain Ropinirole hydrochloride in about 20% over all yield. However, this process is not suitable for large-scale production.

Further, several prior art processes for the preparation of the compound of Formula I disclose the use of highly toxic reagents such as hydrazine hydrate (e.g. EP-B-0266033, EP-B-1242376), cyanide compound (e.g. EP-A-1568689, US-2009/0043111), and nitromethane (e.g. U.S. Pat. Nos. 7,230,118, 7,378,439). Hydrazine hydrate and cyanide compound are extremely harmful and can be fatal when inhaled or absorbed through the skin. Nitromethane is also an energetic high explosive material. These processes are not feasible for large-scale production.

In addition, in Journal of Pharmaceutical and Biochemical Analysis, 2007, 43, 1587-1593 is disclosed that prior art processes for the preparation of compound of Formula I generate a number of structurally similar impurities that are difficult to isolate from the desired drug substance.

Although each of the above patents represents an attempt to overcome the use of costly and hazardous material, there still exists a need for a cost-effective and safer process for large scale production of Ropinirole and the pharmaceutically acceptable salts thereof, which provides higher yield with higher purity.

### SUMMARY OF THE INVENTION

It is, therefore, an object of the present invention to provide an improved process for the preparation of Ropinirole or pharmaceutical acceptable salts thereof or its derivatives, which overcomes the deficiencies of the prior art processes and results to a cost effective industrial production without sacrificing the yield and quality of the product.

Another object of the present invention is to provide an improved method for the preparation of Ropinirole or salts thereof or its derivatives by selecting the appropriate reactants, catalysts, solvent systems and conditions used during the organic reactions, so that the purity (both chemical purity and optical purity) and yield of the reaction are increased and the presence of any contaminants and formed by-products is minimized.

Further object of the present invention is to provide an improved method for the preparation of Ropinirole or salts thereof, or its derivatives, by using milder and safer reaction conditions that helps protecting the environment and the personnel.

In accordance with the above objects of the present invention, a process for the preparation of Ropinirole hydrochloride of Formula I is provided comprising the following steps:

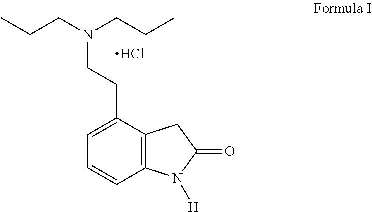

(a) Reaction of N-[2-(2-methyl-3-nitrophenypethyl]-N-propylpropan- 1-amine hydrochloride of Formula II with diethyl oxalate in the presence of sodium alkoxide to obtain ethyl 6-[2-(di-n-propylamino)ethyl]-2-nitrophenyl pyruvate of Formula III;

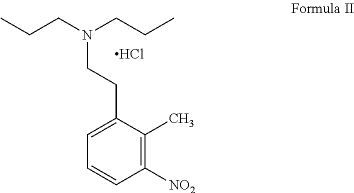

(b) Conversion of ethyl 6-[2-(di-n-propylamino)ethyl]-2-nitrophenyl pyruvate of Formula III to 6-[2-(di-n-propylamino)ethyl]-2-nitrophenyl acetic acid hydrochloride of Formula IV by a one-pot process;

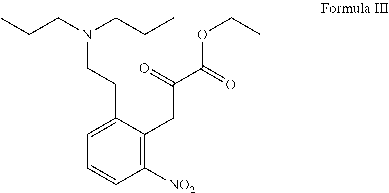

(c) Purification of the compound of Formula IV to obtain substantially pure compound of Formula IV;

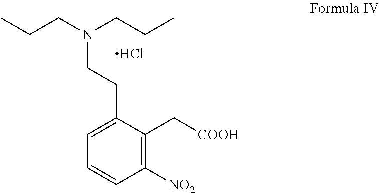

(d) conversion of compound of Formula IV into crude Ropinirole hydrochloride;

(e) removal of impurities; and

(f) recrystallization to obtain crystalline Ropinirole hydrochloride with chemical purity of ≧99.9%.

Preferred embodiments of the present invention are set out in dependent claims 2 to 8.

Other objects and advantages of the present invention will become apparent to those skilled in the art in view of the following detailed description.

### DETAILED DESCRIPTION OF THE INVENTION

The present invention relates to an improved process for the preparation of Ropinirole and pharmaceutically acceptable salts, which is characterized in substantially milder and safer reaction conditions, without sacrificing the yield and quality of the product and low cost of reactants and reagents. The process of the present invention produces highly pure Ropinirole hydrochloride with an improved overall yield of not less than 40% from N-[2-(2-methyl-3-nitrophenyl)ethyl]-N-propylpropan-1-amine hydrochloride. This is attributed to the fine adjustment in reaction and purification conditions

The process of the present invention is safe in operation and benign to the environment. Highly toxic and explosive chemicals, such as hydrazine, cyanide, nitromethane, which constitute the key ingredients in many of the prior art processes have been avoided.

According to the present invention, the process for the preparation of Ropinirole and pharmaceutically acceptable salts thereof comprises the following steps:
a) preparation of a mixture of sodium alkoxide and diethyl oxalate in a mixed solvent of tetrahydrofuran and absolute ethanol;
b) addition of N-[2-(2-methyl-3-nitrophenyl)ethyl]-N-propylpropan-1-amine hydrochloride to the solution obtained from (a) and keeping the reaction mixture at 25-30° C. for 70-72 hours to obtain ethyl 6-[2-(di-n-propylamino)ethyl]-2-nitrophenyl pyruvate;
c) treatment of the ethyl 6-[2-(di-n-propylamino)ethyl]-2-nitrophenyl pyruvate subsequently with sodium hydroxide, hydrogen peroxide, sodium metabisulfite and hydrochloric acid to obtain 6-[2-(di-n-propylamino)ethyl]-2-nitrophenyl acetic acid hydrochloride;
d) purification using methanol followed by acetone to obtain the 6-[2-(di-n-propylamino)ethyl]-2-nitrophenyl acetic acid hydrochloride intermediate in high purity;
e) conversion of the 6-[2-(di-n-propylamino)ethyl]-2-nitrophenyl acetic acid hydrochloride intermediate into crude Ropinirole hydrochloride through reductive amidation;
f) biphasic extraction treatment to remove impurities; and
g) recrystallization in methanol/acetone to obtain substantially pure Ropinirole hydrochloride.

In addition, the present invention provides an efficient method for the removal of structurally similar impurities from Ropinirole. The treatment of removing impurities according to the present invention comprises four consecutive steps.

In the first step, biphasic extraction at pH value between 9.0-9.2 has been proven to be effective to selectively remove the des-N-n-propyl Ropinirole (impurity C) in the aqueous phase.

In the second step, biphasic extraction at pH value equal to 12 selectively removes the N-hydroxy-Ropinirole (impurity A).

In the third step, repeat the biphasic extraction at pH value between 9.0-9.2 removes the remaining des-N-n-propyl Ropinirole (impurity C) in the aqueous phase.

The final step, treatment of the obtained organic phase with sodium hydrosulfite converts the diketo Ropinirole (impurity B) into the targeted molecule Ropinirole.

Thus, the present invention provides highly pure Ropinirole API in improved overall yield.

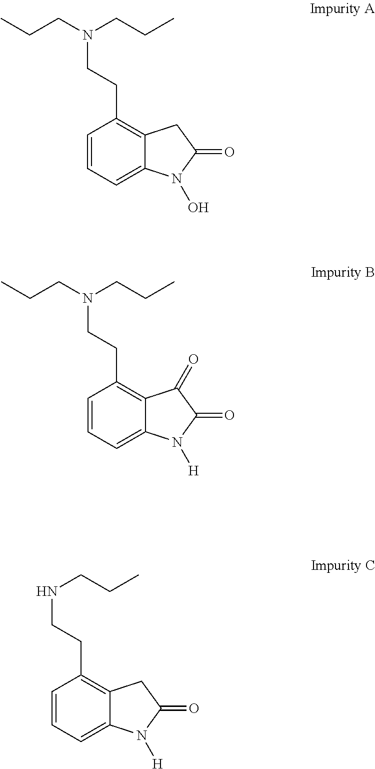

The steps of the process for the preparation of Ropinirole and pharmaceutically acceptable salts thereof according to the present invention will be described in detail.

Stage I. Preparation of 6-[2-(di-n-propylamino) ethyl]-2-nitrophenyl acetic acid hydrochloride

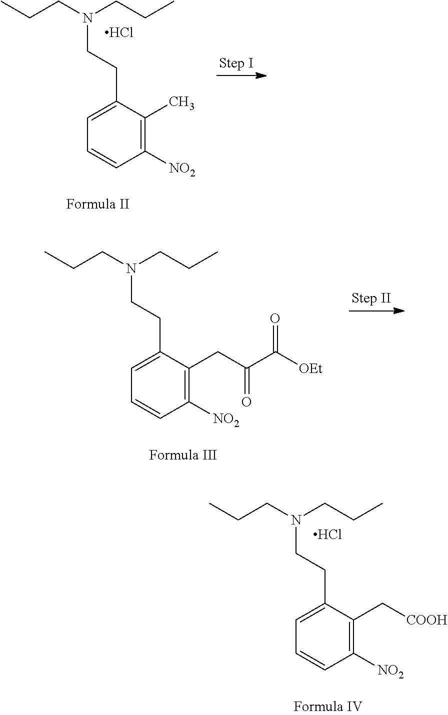

Stage I comprises Step I and II. According to Step I, N-[2-(2-methyl-3-nitrophenyl)ethyl]-N-propylpropan-1-amine hydrochloride, i.e. Formula II, is converted into ethyl 6-[2-(di-n-propylamino)ethyl]-2-nitrophenyl pyruvate, i.e. Formula III. A solution of sodium ethoxide is prepared by carefully adding absolute ethanol into a suspension of sodium metal in anhydrous tetrahydrofuran under inert atmosphere. Subsequently, calculated amount of diethyl oxalate and N-[2-(2-methyl-3-nitrophenyl) ethyl]-N-propylpropan-1-amine hydrochloride (i.e. Formula II) is added to the sodium ethoxide solution. The reaction mixture is maintained at temperature of 25 to 30° C. for 70-72 hours to obtain ethyl 6-[2-(di-n-propylamino)ethyl]-2-nitrophenyl pyruvate (i.e. Formula III).

Further, in Step II ethyl-6-[2-(di-n-propylamino) ethyl]-2-nitrophenyl pyruvate (i.e. Formula III) is converted into 6-[2-(di-n-propylamino) ethyl]-2-nitrophenyl acetic acid hydrochloride (i.e. Formula IV). Compound of Formula III is dissolved in a biphasic solvent mixture containing toluene, methanol and water. NaOH, H₂O₂ and Na₂S₂O₅ are added into the biphasic solution to achieve hydrolyzation, decarboxylation and decoloration with no isolation of the intermediates. The biphasic solution is then filtered and the layers are separated.

The obtained compound is converted into its hydrochloride additional salt form and further it is purified. The salt formation is obtained by adjusting the pH value of the aqueous layer obtained in step II to less than pH 2 using concentrated HCl solution. The purification process includes passing the aqueous solution of crude compound of Formula IV through activated charcoal; treating the filtrate with sodium chloride aqueous solution at temperature of about 65-70° C. then gradually cooling the solution mixture to temperature of about 25-30° C. and filtering. Further purification using methanol and acetone provides compound of Formula IV in high purity.

Stage II: Preparation of Crude Ropinirole Hydrochloride

The preparation of crude Ropinirole hydrochloride from 6-[2-(di-n-propylamino) ethyl]-2-nitrophenyl acetic acid hydrochloride intermediate is achieved by subjecting 6-[2-(di-n-propylamino) ethyl]-2-nitrophenyl acetic acid hydrochloride (Formula IV) to hydrogenation using Pd/C as catalyst and methanol as solvent.

Stage III: Treatment to Remove Impurities

According to the present invention the following process has been developed for the removal of the structurally similar impurities generated during the preparation of Ropinirole. Said process comprises:
(1) dissolving the crude hydrogenation product in a biphasic solvent mixture containing appropriate amount of toluene and water, adjusting the pH value of the aqueous layer to about pH 9 by adding appropriate amount of sodium hydroxide aqueous solution into the biphasic mass and separating the layers and collecting the organic layer. This step has been used to effectively remove impurity C from the crude Ropinirole;
(2) adding designed amount of water to the organic layer obtained from previous step (1) to form a biphasic mass. Bring the pH value of the aqueous layer to above pH 12, separating the layers and collecting the organic layer. This step has been used to selectively remove impurity A from the crude Ropinirole;
(3) the organic layer obtained from previous step (2) is once more mixed with designed amount of water to form a biphasic mass. Adjust the pH value of the aqueous layer to about pH 9 by adding appropriate amount of sodium hydroxide aqueous solution into the biphasic system, separating the layers and collecting the organic layer. This step is used to remove the remaining impurity C;
(4) treating the organic layer obtained from previous step (3) with suitable reducing agent and thereby converting the small quantities of impurity B into the desired compound Ropinirole free base; and
(5) optionally reacting the thus obtained Ropinirole free base with IPA.HCl solution to produce crude Ropinirole hydrochloride essentially free of the process-related structural similar impurities.

Stage IV: Re-crystallization of Ropinirole Hydrochloride

Crude Ropinirole hydrochloride is re-crystallized in a mixture of solvent comprised of methanol and acetone to obtain crystalline Ropinirole hydrochloride with chemical purity above 99.9%.

The process of the present invention will be demonstrated in more details with reference to the following examples, which are provided by way of illustration only and should not be construed as limit to the scope of the reaction in any manner.

### Example 1

### Preparation of 6-[2-(di-n-propylamino) ethyl]-2-nitrophenyl acetic acid hydrochloride

Under nitrogen atmosphere, 38 g of sodium metal (cut into pieces) is suspended in 350 ml of anhydrous tetrahydrofuran (THF) and 350 ml of absolute ethanol is carefully added over a period of 120-180 minutes, while maintaining the temperature below 15° C. and it is stirred for additional 60-90 min. The solution is stirred at about 25-30° C. until all the sodium metal has been dissolved. This will generally take 20-24 hrs. The solution is cooled down to temperature below 15° C. and 225 ml of diethyl oxalate is charged, over a period of 40-70 min, while maintaining the temperature below 15° C. Subsequently, a solution of 100 g of N-[2-(2-methyl-3-nitrophenyl)ethyl]-N-propylpropan-1-amine hydrochloride in 50 ml anhydrous THF and 100 ml absolute ethanol (preheated till dissolved and cooled at 30-35 ° C.) is charged at temperature of about 25-30° C. and the reaction mass is stirred for approximately 70-72 hrs at temperature of about 25-30° C. The solvents are removed by rapid distillation under reduced pressure and maintaining the temperature below 35° C.

1800 ml of dichloromethane is charged to the red oily residue, stirred till dissolution and the reaction mass is cooled to about below 5° C. Add 65 ml of aq. HCl (37% w/w) while keeping the solution temperature below 15° C. (pH=7.0±0.2). Then 1300 ml of D.M. water is added to form a biphasic mass. The biphasic mass is warmed up to temperature 25-30° C. and the biphasic mass is stirred for about 30-45 min. The biphasic mass is filtered through a Celite bed and the wet cake is washed with 300 ml dichloromethane. The organic layer is collected and the solvents are distilled off. Vacuum is applied towards the end of distillation to remove the residue solvent.

To the deep red oily residue, 620 ml of toluene, 45 ml of methanol and 500 ml of D.M. water is added at temperature about 25-30° C. The mixture is stirred to dissolution. 230 ml of aq. NaOH (20% w/v) is added under stirring till pH value is more than pH 12. Then the amount of water required to have total volume of water added from the 2 last steps equal to 780 ml is added. The reaction mass is stirred for 90-120 min and cooled down to temperature of about 0-5° C. Subsequently, 44 ml of aq. H₂O₂ (30% w/w) is charged over a period of 15 min and further the reaction mass is stirred for additional 5-10 min by maintaining the temperature below 15° C. A solution of 22 g Na₂S₂O₅ in 44 ml D.M. water is formed and added to the reaction mass for a period of 5-10 min while maintaining the temperature below 15° C., and stirring continuously for 45-60 min while keeping temperature below 15° C. 65 ml aq. NaOH (50% w/v) is added till pH value is more than pH 12, allowing the reaction mass to warm up to 25-30° C. The biphasic reaction mass is filtered through filter paper and separate the layers.

About 110 ml of aq. HCl (37%, w/w) is added to the aqueous layer till pH value is less than 2, while maintaining the temperature at about 25-30° C. The reaction mass is heated to temperature of about 65-70° C. and stirred until dissolution. 25 g of activated charcoal is charged under stirring. Continue heating the reaction mass with stirring at temperature of about 65-70° C. for an additional period of 45-75 min. The hot suspension is filtered through a Celite bed and the solids are washed with 45 ml D.M. water. The filtrate is heated to temperature of 65-70° C. and 125 g NaCl is added to the solution. The reaction mass is stirred for additional 15-30 min while maintaining the temperature at about 65-70° C. The liquid is gradually cooled to about 25-30° C. over a period of 3-4 hrs and it is let stand at such temperature for additional 45-60 min. The solid is filtered and the wet cake is dried in vacuum oven at 60° C. till LOD≦5%. The yield obtained is about 70-90 g.

In a 250 ml 3-neck round-bottom flask, the mass obtained from the previous step is added to 160 ml of MeOH with stirring. The mixture is heated to 65-70° C. till a homogenous mass is formed and stirring is continued for additional 10-15 min. The hot suspension is filtered through filter paper and slurry wash with 80 ml MeOH. MeOH is distilled off under atmospheric pressure. 160 ml Acetone is added to the residue and the mixture is heated at temperature of 45-50° C. to form a homogenous mass and stirring is continued at the same temperature for additional 30-60 min. The suspension is cooled slowly to temperature of 25-30° C. over a period of 90-180 min. Then it is further cooled to 0-5° C. over a period of 30 min. Stirring is continued for additional 90 to 120 min at temperature of 0-5° C. This suspension is filtered and the solids are washed with 80 ml chilled Acetone. The wet cake is dried in vacuum oven at 60° C. till LOD≦1%. The yield obtained is about 65-80 g.

### Example 2

### Preparation of Crude 4-[2-(di-n-propylamino) ethyl]-1, 3-dihydro-1H-indol-2-one hydrochloride

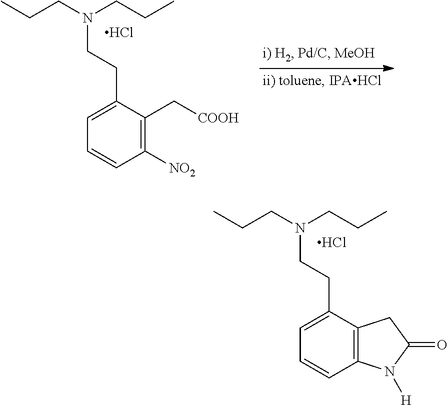

100 g of 6-[2-(di-n-propylamino) ethyl]-2-nitrophenyl acetic acid hydrochloride is dissolved in 1500 ml methanol and the solution is charged to a high pressure reactor. A suspension of 20 g of Pd/C 5% (50% wet) in 50 ml methanol is added (wash the flask which contained the suspension with 50 ml methanol) and the reactor is sealed. Hydrogen gas is charged at temperature of about 25-35° C. until the pressure reaches 2.8-4.4 kg/cm² and the mixture is stirred for about 2-4 hrs. The catalyst is filtered and washed with 200 ml methanol. The filtrate is evaporated to dryness (apply vacuum at the end of distillation).

The residue is charged to 1000 ml D.M. water and 2000 ml toluene. About ˜18 ml aq. NaOH (50% w/v) is added to adjust the pH value of the aqueous layer to pH 9.0-9.2. The mixture is stirred for 10-20 min and the layers are separated. The aqueous layer is extracted with 2000 ml toluene. The two organic layers are combined and 1000 ml of D.M. water is added. The pH value of the aqueous layer is brought to above pH 12 using about ˜4 ml of aq. NaOH (50% w/v). The mixture is stirred for 10-20 min and the layers are separated. 1000 ml D.M. water is added to the organic layer and the pH value of the aqueous layer is adjusted to pH 9.0-9.2 using about ˜1 ml aq. HCl (37% w/w). The mixture is stirred for 10-20 min and the layers are separated.

1000 ml D.M. water and 100g of Na₂S₂O₄ (or Na₂S₂O₃ or Na₂S₂O₅) are added to the organic layer and the reaction mass is stirred for 60-90 min. The pH value of the aqueous layer is brought to above pH 10 using about ˜18 ml aq. NaOH (50% w/v). The reaction mass is stirred for 10-20 min and the layers are separated. The organic layer is concentrated by distillation under reduced pressure until the volume of the reaction mass is approximately 1 L. The mass is passed through a celite bed to filter off the inorganic particles followed by rinse the flask with 100 ml toluene. The filtrate is concentrated to dryness by distillation under reduced pressure.

The oily residue is dissolved in 300 ml toluene and 64 ml of IPA.HCl (15-25% w/v) is added while stirring at temperature of 25-35° C. The mass is cooled gradually to 0-5° C. over a period of time 90-150 min and the reaction mass is stirred continuously for additional 2-3 hours. The suspension is filtered through a Buchner funnel, slurry washed with 100 ml toluene, and the wet cake is suck dry for 45-60min. Then, the wet cake is dried in vacuum oven at temperature of 50-55° C. till LOD <0.5%. The yield (Ropinirole hydrochloride crude) obtained is 60-75 g.

### Example 3

### Purification of 4-[2-(Di-n-propylamino) ethyl]-1, 3-dihydro-1H-indo1-2-one hydrochloride (Ropinirole Hydrochloride)

100 g of crude 4-[2-(di-n-propylamino) ethyl]-1, 3-dihydro-1H-indol-2-one hydrochloride is added into 375 ml methanol. The mixture is degassed at temperature of 25-30° C. and heated to 64-66° C. with stirring until dissolution. Then, the mixture is cooled to temperature of 60-63° C. and filtered under inert atmosphere. The filtrate is re-heated to 60-63° C. and 200 ml acetone is added over a period of 5-15 min, maintaining the solution temperature at 55-60° C. The mass is cooled to 0-5° C. over a period of 60-120 min and stirring is continued for 60-90 minutes.

The suspension is filtered through Buchner funnel and slurry-washed with 200 ml Acetone. The wet cake is dried in vacuum oven at 50-55° C. till LOD≦0.5%. The yield obtained is 80-90 g.

The present invention describes a large-scale manufacture process for the preparation of Ropinirole hydrochloride in high purity at relative low production cost compared to the available processes for producing similar products.

Therefore, the Ropinirole hydrochloride according to the process of the present invention is obtained in excellent yield (above 40%) and in high purity (above 99.9%).

While the present invention has been described with respect to the particular embodiments, it will be apparent to those skilled in the art that various changes and modifications may be made in the invention without departing from the spirit and scope thereof, as defined in the appended claims.

1. A process for the preparation of Ropinirole hydrochloride of Formula I, which comprises 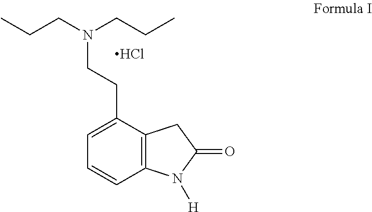
(a) Reaction of N-[2-(2-methyl-3-nitrophenypethyl]-N-propylpropan-1-amine hydrochloride of Formula II with diethyl oxalate in the presence of sodium alkoxide to obtain ethyl 6-[2-(di-n-propylamino)ethyl]-2-nitrophenyl pyruvate of Formula III; 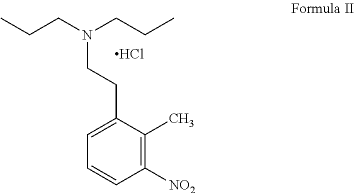
(b) Conversion of ethyl 6-[2-(di-n-propylamino) ethyl]-2-nitrophenyl pyruvate of Formula III to 6-[2-(di-n-propylamino) ethyl]-2-nitrophenyl acetic acid hydrochloride of Formula IV by a one-pot process; 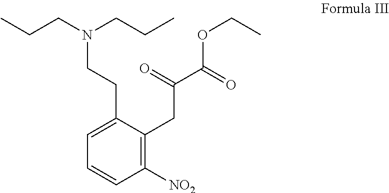
(c) Purification of the compound of Formula IV to obtain substantially pure compound of Formula IV; 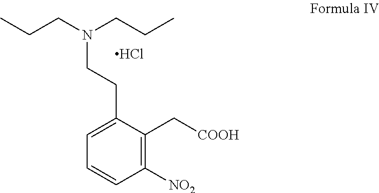
(d) conversion of compound of Formula IV into crude Ropinirole hydrochloride;
(e) removal of impurities; and
(f) recrystallization to obtain crystalline Ropinirole hydrochloride with chemical purity of≧99.9%.

2. The process according to claim 1, wherein the reaction of step (a) is performed by adding a solution of Formula II N-[2-(2-methyl-3-nitrophenyl) ethyl]-N-propylpropan-1-amine hydrochloride to a mixture of diethyl oxalate and sodium ethoxide in tetrahydrofuran containing solvent and keeping the reaction mixture at temperature of about 25-30° C. for a period of time about 70-72 hours.

3. The process according to claim 1, wherein the one-pot process of conversion of the compound of Formula III into compound of Formula IV in step (b) comprises: dissolving compound of Formula III in a biphasic solvent mixture containing toluene, methanol and water; adding NaOH, H₂O₂ and Na₂S₂O₅ into the biphasic solution to achieve hydrolyzation, decarboxylation and decoloration respectively in one pot; filtering the biphasic solution and separating the layers; adjusting the pH value of the aqueous layer to below pH 2 using concentrated HCl solution; and obtaining compound of Formula IV.

4. The process according to claim 1, wherein the purification of step (c) is recrystallization in methanol followed by acetone.

5. The process according to claim 1, wherein the conversion of step (d) comprises hydrogenating compound of Formula IV using Pd/C as catalyst and methanol as solvent.

6. The process according to claim 1, wherein the removal of impurities of step (e) comprises:
dissolving crude Ropinirole in a biphasic mixture comprising toluene and water
adjusting the pH of the aqueous phase to about pH 9;
separating the organic layers and treating the organic layer obtained with an aqueous solution and adjusting the pH value of the aqueous phase to pH above 12;
separating the organic layers and treating the organic layer obtained with an aqueous solution and adjust the pH value of the aqueous phase to about pH 9, followed by separating the layers;
treating the organic layer with reducing agent; and
optionally, converting the purified Ropinirole base into Ropinirole Hydrochloride.

7. The process according to claim 6, wherein the reducing agent is selected from a group comprising Na₂S₂O₄, Na₂S₂O₃ and Na₂S₂O₅.

8. The process according to claim 1, wherein the recrystallization of step (f) comprises: addition of crude Ropinirole hydrochloride into methanol and heat at 64-66° C. until dissolution; filtration of the hot solution under inert atmosphere; addition of acetone to the filtrate at temperature of 55-60° C.; cooling the solution to temperature of about 0-5° C. to facilitate crystallization; and obtaining crystalline Ropinirole hydrochloride with chemical purity >99.9%.
